(19) United States
(12) Patent Application Publication
(10) Pub. No.: US 2012/0073005 A1
(43) Pub. Date: Mar. 22, 2012

(54) TRANSGENIC ANIMAL CARRYING AT LEAST TWO TYPES OF FOREIGN FUNCTIONAL RNA

(75) Inventors: Hiroyuki Watanabe, Takasago-shi (JP); Maki Masutani, Takasago-shi (JP); Kenji Kyogoku, Takasago-shi (JP)

(73) Assignee: KANEKA CORPORATION, Osaka-shi, Osaka (JP)

(21) Appl. No.: 13/266,721

(22) PCT Filed: Apr. 28, 2010
(86) PCT No.: PCT/JP2010/003051

§ 371 Date: Dec. 7, 2011

(30) Foreign Application Priority Data
Apr. 28, 2009 (JP) PCT/JP2009/058410

(51) Int. Cl. A01K 67/027

(52) U.S. Cl. 800/19; 800/13

(57) ABSTRACT

The present invention relates to a transgenic animal which intracellularly expresses at least two types of foreign functional RNA and has resistance to a pathogen such as a virus. In the animal carrying multiple types of foreign functional RNA, the proliferation of a pathogen can be more significantly inhibited compared to an animal carrying one type of foreign functional RNA.

### TECHNICAL FIELD

The present invention relates to a transgenic animal intracellularly expressing at least two types of foreign functional RNA and having resistance to a pathogen.

### BACKGROUND ART

Domestic animals are important industrial animals for human life. However, some infectious diseases transmitted by domestic animals cause enormous damages on domestic animals and humans.

Foot-and-mouth disease, rabies, classical swine fever and the like in mammals are highly infectious, and once these diseases occur, only possible way to address the problem is to contain the disease by killing and incinerating all domestic animals that have lived with infected animals and have possibly contacted such animals, and by disinfecting the surrounding area.

Many pathogens are known in domestic poultry which are transmitted among birds. Among them, influenza viruses are the pathogens which are transferred to not only birds but also humans or domestic animals and cause severe infectious diseases. The humans have developed anti-influenza drugs and vaccines in order to combat such influenza viruses. However, prevention measures against evolving, unknown novel influenza viruses have not yet developed sufficiently. It has been known that influenza viruses may mutate by being repeatedly transmitted among birds, or between birds and other animals, or between birds and humans. Thus, therapeutic agents and vaccines may not be effective in some cases.

It is very important to obtain an animal which is resistant to a pathogen. Breeding and gene recombination are conceived as methods of producing such an animal.

An exemplary method by breeding has been known in which repeated mating is carried out between chicken lineages genetically having resistance to a pathogen, in order to obtain a chicken species having enhanced resistance against the pathogen. More specifically, production of chickens resistant to influenza and to Marek's disease has been attempted. However, this method of producing resistant chickens requires extremely longtime, and it has also been pointed out that the thus produced resistant chickens become a carrier of the pathogen due to their resistance, resulting in transfer of the pathogen to other animals. Moreover, the resistance is not always sufficiently exerted when the pathogen is mutated.

An exemplary method by gene recombination has been known in which recombinant technologies using various vectors and RNA interference (RNAi) technologies (Non-patent document 1) are combined to produce an animal which is resistant to a specific pathogen (or inhibits proliferation of the pathogen) (Patent documents 1 and 2). According to an example to which this method has been applied, production of chickens has been attempted into which an shRNA (short hairpin RNA) has been introduced that inhibits replication of avian influenza virus (Patent document 3). However, again, there is a problem in resistance when the pathogen is mutated.

Patent document 1: WO 2005/003348

Patent document 2: WO 2006/102461

Patent document 3: US Patent Application Publication No. 2008-0222743

Non-patent document 1: Fire et al., Nature 391, 806-811 (1998)

### SUMMARY OF THE INVENTION

An object of the present invention is to produce a transgenic animal which has sufficient resistance even against a mutated pathogen.

The present inventors have solved the above problems after extensive studies by producing a transgenic animal intracellularly expressing at least two types of foreign functional RNA. More specifically, they have used domestic poultry as an example and produced a chicken expressing more than one foreign functional RNA to accomplish the present invention.

Thus, the present invention provides the followings:

(1) a transgenic animal intracellularly expressing at least two types of foreign functional RNA and having resistance to a pathogen;

(2) the transgenic animal according to (1), wherein the functional RNA is one or more polynucleotides (RNAs) selected from shRNAs, miRNAs, ribozymes, siRNAs and aptamers;

(3) the transgenic animal according to (1) or (2), wherein the functional RNA is a RNA having a base sequence having 50% or more sequence identity with a complementary strand of a partial genomic base sequence of the pathogen;

(4) the transgenic animal according to any of (1) to (3). wherein the resistance to the pathogen corresponds to inhibition of proliferation of the pathogen to 50% or less; (5) the transgenic animal according to any of (1) to (4), wherein the pathogen is a virus;

(6) the transgenic animal according to (5), wherein the virus is an influenza virus;

(7) the transgenic animal according to any of (1) to (6), wherein the transgenic animal is a bird;

(8) the transgenic animal according to (7), wherein the bird is a domestic poultry; and

(9) the transgenic animal according to (8), wherein the domestic poultry is a chicken.

According to the present invention, a transgenic animal having superior resistance against a pathogen can be produced, so that enormous damages to domestic animals and humans due to infectious diseases transmitted by domestic animals can be prevented. For example, in the case of domestic poultry, typically chickens, the following two effects may be mentioned; first, transfer of a zoonosis, typically avian influenza, among birds and from birds to humans can be prevented and pandemic of a terrifying virus unknown to humans, typically novel influenza, can be prevented. Secondly, it is possible to prevent spreading a pathogenic virus among birds by conferring resistance against the pathogen to domestic poultry, and to reduce enormous economic loss at poultry farms and the like.

### BRIEF DESCRIPTION OF THE DRAWINGS

FIG. 1 is a graph of virus titer and hatching rate (survival rate) compared between single and multiple shRNA expressions;

FIG. 2 is a schematic view of an transfer vector for verifying the pathogenic virus replication inhibition effect of shRNAs expressed in animal cells; and

FIG. 3 is a graph indicating the pathogenic virus replication inhibition effect in chicken cells expressing one type of shRNA (NP or M2) or two types of shRNA (NP and M2).

### BEST MODE FOR CARRYING OUT THE INVENTION

Preferred embodiments in the present invention are described hereinafter. However, the scope of the present invention is not limited to the description.

The present invention is characterized in that a transgenic animal having resistance to a pathogen is produced by the following steps:
(1) selecting functional RNAs that inhibit proliferation of a pathogen;
(2) constructing a vector capable of expressing more than one type of functional RNA according to (1) in an animal cell; and
(3) producing the transgenic animal using the vector of (2).

The respective steps are now described in detail.

(1) Functional RNAs for inhibiting proliferation of a pathogen (e.g. a pathogenic virus) are selected. Examples of the pathogenic virus include RNA viruses and DNA viruses. The pathogenic virus may be a pathogenic virus that is transmitted by domestic animals and the like. Examples thereof include foot-and-mouth disease, rabies, classical swine fever and the like. Pathogenic viruses transmitted by domestic poultry or wild birds or mutants thereof, in particular pathogenic viruses transmitted by chickens or mutants thereof are also preferred as targets.

Examples of such pathogenic viruses may include influenza viruses, particularly avian influenza viruses, more specifically H5N1 avian influenza virus or mutants thereof. These influenza viruses are RNA viruses classified to Orthomyxoviridae family. Besides influenza viruses, other examples include viruses transmitted by domestic poultry, and mutants thereof, such as Newcastle disease virus, chicken infectious bronchitis virus, chicken leukosis virus, chicken encephalomyelitis virus, chicken nephritis virus, chicken infectious laryngotracheitis virus, reticuloendotheliosis virus, Marek's disease virus, infectious bursal disease virus, avian reovirus, avian adenovirus, EDS-76 virus, chicken anemia virus, turkey rhinotracheitis virus, avian paramyxovirus, and fowlpox virus; and viruses that can be transmitted by any birds including water birds and wild birds, and mutants thereof.

Among others, emphasis may be on avian influenza virus, Marek's disease virus, Newcastle disease virus or the like in terms of its broad spread among domestic poultry and enormous scale of damages caused thereby, and particularly on avian influenza in terms of possibility of transmittance to humans.

Examples of the animal according to the present invention include mammals and birds which may be bred by humans, for example, mammals such as cows, horses, sheep, goats, pigs, dogs, and cats; and birds (domestic poultry) such as chickens, quails, turkeys, geese, wild ducks, domestic ducks, and ostriches.

The functional RNA for inhibiting proliferation of a pathogen as used herein denotes a functional RNA which comprises a RNA having a base sequence complementary to a partial genomic base sequence of the target pathogen. The partial genomic base sequence has 5 bases or more, preferably 10 bases or more, more preferably 15 bases or more, and particularly preferably 20 bases or more. A functional RNA which comprises a RNA consisting of a base sequence complementary to such a partial base sequence can be suitably used for the present invention. The functional RNA suitable to be used for the present invention may be one that comprises a RNA consisting of a base sequence having 50% or more, preferably 60% or more, more preferably 70% or more, even more preferably 80% or more, particularly preferably 90% or more, and most preferably 100% sequence identity with a base sequence complementary to a partial genomic base sequence of the target pathogen.

The partial genomic base sequence of the target pathogen is preferably a region having low frequency of mutation. As used herein, the region having low frequency of mutation means a region in which DNA or RNA genomic base sequences between different strains of a pathogen are 80% or more, preferably 85% or more, more preferably 90% or more, and most preferably 100% identical. Regions contributing to replication and structure maintenance of a pathogen may be targeted. For example, influenza A virus has 8 gene regions, and among the regions the regions that replicate the RNA genome of the virus called PA, PB1, PB2 and NP and the gene region that contributes to structure maintenance of the virus called M may be suitable targets.

The functional RNA expressed in an animal cell is not limited in terms of its length and structure so long as it has an inhibitory function on proliferation of the target pathogen. The animal cell used herein means a cell derived from animal such as bird or mammal. A collection of the cells is called an animal.

As used herein, the inhibitory function on proliferation of a pathogen means that in a cell or animal to which the functional RNA has been introduced, the growth rate of the pathogen is inhibited to 50% or lower, preferably 40% or lower, and more preferably 30% or lower compared to a non-manipulated control cell or animal. The growth rate of a virus can be measured by any known methods such as serum aggregation test, hemagglutination inhibition test, neutralization reaction, agar-gel precipitation test, antigen-antibody reaction, fluorescence antibody technique, quantitative PCR.

The functional RNA in the present invention may be suitably a functional RNA that may adopt a secondary or tertiary conformation such as a loop or hairpin. Examples of such a functional RNA may include, for example, shRNAs, siRNAs, miRNAs, ribozymes and aptamers. shRNAs may be subjected to processing in cells and exist as short double-stranded RNA dimers (siRNAs), which may associate with biomolecules such as proteins or lipids to form complexes. shRNAs, siRNAs and the like are preferably used for the present invention because they may frequently induce RNA interference, and can significantly degrade and inhibit the genome of the target pathogen.

RNA interference is a phenomenon in which a single- or double-stranded RNA sequence having a sequence that is complementary in some extent to a genomic base sequence of a target pathogen forms a protein-RNA complex called a RNA-induced silencing complex (RISC) in a cell to cleave the genome of the target pathogen.

A RNA sequence having high proliferation inhibition effect against a certain pathogen can be selected according to any known test methods such as in vitro tests, for example by using a target pathogen or the genome thereof in cultured cells; in vivo tests such as animal infection tests; or ex vivo tests using cells isolated from an animal expressing the functional RNA, or according to literature information.

It is preferable that at least two types of functional RNA are expressed in an animal cell. By expressing more than one type of functional RNA, the resistance to a pathogen having high frequency of mutation can be maintained.

(2) A vector is prepared which can introduce into an animal cell at least two types of functional RNA gene for inhibition of proliferation of a pathogen of various types described above. The vector may be a plasmid DNA or a double-stranded DNA fragment as long as it can be introduced into an animal cell of interest. The plasmid DNA as used herein denotes a circular double-stranded DNA. The vector is introduced into an animal cell by a known gene introduction technique such as calcium phosphate method, lipofection, or electroporation. The thus introduced vector is preferably incorporated into the chromosome of the animal cell. The term chromosome as used herein means a genomic DNA which is replicated during cell division and inherited to a daughter cell.

Known vectors may be used in order to effectively incorporate the functional RNA gene into a chromosome, and examples thereof include, for example, virus vectors (retrovirus vectors etc.). Examples of the retrovirus vectors include, but not limited to, retrovirus vectors derived from Moloney murine leukemia virus, Moloney murine sarcoma virus, avian leukosis virus (ALV), murine stem cell virus (MSCV), murine embryonic stem cell virus (MESV) and the like; and lentivirus vectors derived from human immunodeficiency virus (HIV) and the like.

In order to express the functional RNA that inhibits proliferation of a pathogen in an animal cell, a vector is constructed in which the functional RNA is incorporated in an expression cassette. The vector to be used is not particularly limited, but preferably has a suitable design for expression of the functional RNA in a cell or in an animal which is a collection of cells, including a promoter, an enhancer, a regulatory factor and the like, in addition to the functional RNA sequence.

A promoter is a DNA sequence that determines a transcription initiation site of a gene and acts to directly regulate the frequency of transcription. The promoter is not limited as long as it effectively functions in expression of RNA. Pol III promoters such as U6 promoter and H1 promoter, which are conventionally used for RNA expression, are suitable for expression of short RNA. Alternatively, pol II promoters such as virus promoters, for example, EF1α promoter, thymidine kinase promoter, simian virus 40 (SV40) promoter, murine phosphoglycerokinase (PGK) promoter, cytomegalovirus (CMV) promoter, Rous sarcoma virus (RSV) promoter and the like, promoters of animal origin such as chicken beta-actin promoter and inducible promoters such as tetracycline-inducible promoter may also be used. Expression can be effected in a specific tissue or cell by using a tissue-specific promoter. Promoters that function in mucous membranes and digestive organs are particularly preferable.

An enhancer is a sequence that facilitates transcription from a promoter. Any combination with a promoter without limitation is possible. Examples of the enhancer include, but not limited to, SV40, CMV, thymidine kinase enhancer, steroid response element, lysozyme enhancer and the like.

A regulatory factor is a DNA sequence that contributes to transcription regulation and stabilization of transcribed RNA. The regulatory factor is not particularly limited, and may be a woodchuck post-transcriptional regulatory element (WPRE; see U.S. Pat. No. 6,136,597) or the like.

The thus prepared vector can express more than one type of functional RNA in an animal cell by carrying at least two expression units containing a promoter and/or an enhancer. Alternatively, it has been known that the same effect can be obtained by subjecting an RNA transcribed from one expression unit to intracellular processing to cause expression of at least two types of functional RNA, as a result. Alternatively, it is also possible to introduce into an animal cell at least two types of vector each of which is capable of expressing one type of functional RNA in an animal cell so as to co-express multiple types of functional RNA in the cell. When a vector carries a plurality of expression units, the expression units may be tandemly linked in the same direction, or may be arranged bidirectionally.

The vector used may be, but not limited to, a virus vector or the like. When a virus vector is used, its titer is preferably high such as, when it is measured with NIH3T3 cells or Hela cells, 1×10⁶ cfu/ml or more, preferably 1×10⁷ cfu/ml or more, more preferably 1×10⁸ cfu/ml or more, even more preferably 1×10⁹ cfu/ml or more, and in particular 1×10¹⁰ cfu/ml or more.

(3) A procedure is now described in detail by which a cell or animal showing resistance to a pathogen is produced by using the above vector. When a cell showing resistance to a pathogen is to be produced, the vector may be introduced into a cell with a known procedure. When an animal showing resistance to a pathogen is to be produced, a known procedure can be used for e.g. mammals in which the vector is introduced into a fertilized egg or early embryo prior to the implantation into the uterus of a host animal, and then the egg or embryo is allowed to be developed as an individual organism. A known procedure for birds may also be used in which, by using a sperm egg, the vector is introduced into an early embryo and then the embryo is allowed to be hatched. Accordingly, a cell or animal intracellularly expressing at least two types of functional RNA can be obtained.

The copy number (average) of a gene introduced in the thus produced cell or animal intracellularly expressing at least two types of functional RNA (such as shRNA) is generally 0.01 or more, preferably 0.1 or more, more preferably 1 or more, and even more preferably 2 or more.

Any known procedures for evaluation of the thus produced cell or animal intracellularly expressing at least two types of functional RNA (such as shRNA) can be used. The evaluation can be carried out by in vitro tests in which expressed functional RNA sequences are detected, in vivo viral resistance tests and the like.

According to an in vitro test, RNA is extracted from the produced animal cell intracellularly expressing at least two types of functional RNA and the target RNAs can be detected by Northern blotting or quantitative or semi-quantitative real-time PCR.

According to an in vivo test, the resistance to a pathogenic virus can be evaluated by allowing the produced animal intracellularly expressing at least two types of shRNA to be infected by the pathogenic virus and measuring the survival rate or production of antibodies.

Alternatively, it is also possible to experimentally check the virus replication inhibition effect of the functional RNA without using a pathogenic virus; for example, the extent of inhibition of reporter gene expression may be determined. Here, the reporter gene is designed to be inhibited in its expression by the functional RNA. The inhibition of reporter gene expression is quantified in the cell expressing the functional RNA by a known technique such as real-time PCR. Alternatively, the following procedure is also possible: a cell expressing a reporter gene is produced, the gene capable of expressing the functional RNA is introduced into the cell and the extent of inhibition of reporter gene expression is then determined by a known technique such as real-time PCR.

Of course, the produced animal intracellularly expressing at least two types of functional RNA can be inbred or outbred with a wild-type animal in order to produce an offspring having superior resistance against the pathogen.

### EXAMPLES

The present invention is now described in detail referring to the examples, which do not limit the present invention. Unless otherwise stated, procedures for gene manipulation and the like were carried out according to typical methods (J. Sambrook, E. F. Fritsch, t. Maniatis; Molecular Cloning, A Laboratory Manual, 2nd Ed, Cold Spring Harbor Laboratory). Unless otherwise stated, cell culture was carried out according to typical methods (Hideki Koyama (Ed.), “Saibo Baiyo Labo Manual (Laboratory Manual for Cell Culture) ”, Springer-Verlag Tokyo, 1999). Unless otherwise stated, products specified by trade names or names of manufacturers were used according to instructions provided with the products. The term “vector” or “vector construct” used herein means a circular double-stranded DNA (so called plasmid vector). A replication-incompetent virus for gene introduction was used as “retrovirus vector”.

### EXAMPLE 1

### Construction of shRNA Expression Vector Construct

Expression vectors expressing shRNAs having H5N1 avian influenza virus inhibition effect were constructed.

1-1: Synthesis of shRNAs

DNA sequences encoding shRNAs which respectively target NP and M2 of influenza A virus were synthesized. SEQ ID NOs: 1 and 2, respectively, were synthesized (SIGMA Genosys). As a control, a DNA sequence (SEQ ID NO: 3) encoding an shRNA which targets GFP was synthesized (SIGMA Genosys).

### 1-2: Synthesis of hU6 Promoter

A human U6 promoter sequence was synthesized as a pol III promoter. A DNA sequence of SEQ ID NO: 4 was synthesized (SIGMA Genosys).

1-3: Construction of shRNA Expression Vector Construct

The SEQ ID NO: 4 was digested with restriction enzymes EcoRI and BamHI, migrated in a 3% agarose gel by electrophoresis and stained with ethidium bromide before the bands at about 300 bp were excised with a cutter knife. DNA was then purified from the agarose gel with QIAEXII (QIAGEN) and eluted in 10 μl TE to prepare an insert. A retrovirus vector construct (1 μg) based on the pMSCVneobactfEPOwpre vector disclosed in the patent document (Example 6 in JP 2007-89578 A) was digested with restriction enzymes EcoRI and BamHI, migrated in a 1% agarose gel by electrophoresis and stained with ethidium bromide before the bands at about 6000 bp were excised with a cutter knife. DNA was then purified from the agarose gel with QIAEXII (QIAGEN) and eluted in 10 μl TE to prepare a vector. To a mixture of 1 μl each of the insert and the vector were added 2 μl MilliQ water and 4 μl SolutionI (DNA Ligation Kit Ver2.1; TAKARA) for thorough mixing, and the ligation reaction was carried out at 16° C. for 30 minutes. Escherichia coli (E. coli) DH5α (TAKARA) was transformed with 1 μa of the reaction solution (heat-shock at 42° C. for 45 seconds after mixing) and allowed to form colonies on a LB agar plate containing ampicillin. On the next day, a single colony was picked up and cultured in a LB liquid medium at 37° C. for 16 hours. The culture supernatant was collected and then the plasmid was extracted (QIAprep Spin Miniprep Kit; QIAGEN). This plasmid was designated as pMSCVneoU6wpre. In the similar manner as above, the DNA fragments of SEQ ID NOs: 1, 2 and 3 were digested with the restriction enzymes, DNAs were purified to prepare inserts, and the inserts were incorporated downstream of the hU6 promoter of the pMSCVneoU6wpre to give vectors pMSCVneoU6NPwpre, pMSCVneoU6M2wpre and pMSCVneoU6GFPwpre, respectively.

The regions of hU6 and NP were amplified by PCR using the pMSCVneoU6NPwpre as a template. The thus obtained DNA fragment of about 300 bp was designated as U6NP.

The pMSCVneoU6M2wpre and the U6NP were used as a vector and an insert, respectively, for ligation to construct a retrovirus vector construct into which two shRNA expression units were incorporated, pMSCVneoU6NPU6M2wpre.

### EXAMPLE 2

### Preparation of Retrovirus Vector

### 2-1: Preparation of Transient Retrovirus

The vector construct prepared in Example 1 (pMSCVneoU6NPwpre) was used to transform E. coli DH5α (TAKARA), and a single colony was cultured in a LB medium (100 ml) while shaking at 37° C. for 16 hours before the cells were collected by centrifugation and purified to be endotoxin-free (EndoFree-plasmid Maxi Kit: QIAGEN).

GP293 cells (Clontech) were seeded on a 100-mm collagen-coated culture dish (IWAKI) at 5×10⁶ cells and cultured in D-MEM High-Glucose (GIBCO) containing 10% FBS for 24 hours prior to transfection of the above vector construct and pVSVG plasmid (Clontech) (24 μg each) with LipofectAMINE 2000 (Invitrogen). Conditions of the transfection were in accordance with the instruction attached to LipofectAMINE 2000. After 6 hours, the culture supernatant was aspirated from the culture dish, 9 ml fresh D-MEM containing 10% FBS and 200 μl 1M HEPES Buffer Solution (GIBCO) were added, and the culture was continued for additional 24 hours.

The culture supernatant was passed through a 0.45-μm cellulose acetate filter (ADVANTEC) and the filtrate was collected in a centrifuge tube. An ultracentrifuge CS100GXL (Hitachi Koki Co., Ltd.) was used for ultracentrifugation (50000 G, 1.5 hours, 4° C.) to concentrate the transiently expressed retrovirus. The retrovirus concentrate was suspended in 20 μl TE to obtain a transient retrovirus preparation.

### 2-2: Establishment and Selection of Packaging Cell Line

GP293 cells were seeded on a 96-well collagen-coated plate (IWAKI) to be 1×10⁴ cells/well, and on the next day, polybrene was added at a final concentration of 8 μg/ml before 20 μl of the transient retrovirus preparation prepared in 2-1 was added for infection. The infected GP293 cells were limiting diluted with a D-MEM medium to 0.5 cells/well on the 96-well collagen-coated plate (IWAKI). D-MEM High-Glucose (GIBCO) containing 10% FBS to which G418 (SIGMA) had been added to a final concentration of 1 mg/mg was used for selection by the drug. The medium was exchanged once three days, colony formation was observed in the plate, and a clone having high cell growth rate was selected as a packaging cell line (pMSCVneoU6NPwpre/GP293).

A packaging cell line for the vector construct pMSCVneoU6NPU6M2wpre prepared in Example 1 was also selected in the similar manner as above, which was designated as pMSCVneoU6NPU6M2wpre/GP293.

### 2-3: Preparation of Retrovirus Vector Having High Titer

The packaging cell line (pMSCVneoU6NPwpre/GP293) prepared in 2-2 was seeded on a 100-mm collagen-coated dish (IWAKI) at 5×10⁶ cells and cultured overnight (D-MEM medium containing 10% FBS). Transfection of plasmid pVSVG (24 μg) was carried out using LipofectAMINE 2000 and after 6 hours, the medium was exchanged (D-MEM medium containing 10% FBS) . HEPES Buffer Solution (200 μl, 1 M, GIBCO) was added and culture was carried out for additional 24 hours.

The culture supernatant was passed through a 0.45-μm cellulose acetate filter (ADVANTEC) and the filtrate was collected in a centrifuge tube. An ultracentrifuge CS100GXL (Hitachi Koki Co., Ltd.) was used for ultracentrifugation (50000 G, 1.5 hours, 4° C.) to concentrate the transiently expressed retrovirus. The retrovirus concentrate was suspended in 20 μl TE and used as a concentrated virus solution in the injection experiment in 2-4. The packaging cell line pMSCVneoU6NPU6M2wpre/GP293 was also subjected to the similar procedure to prepare a retrovirus vector having high titer in order to carry out microinjection into fertilized chicken eggs.

### 2-4: Titration of Retrovirus Vector

NIH3T3 cells (ATCC) were seeded in a 6-well plate (IWAKI) to 1.5×10⁴ cells/well, and after 24 hours the medium was exchanged (D-MEM medium containing 10% FBS (GIBCO) to which polybrene (Sigma) was added to a final concentration of 8 μg/ml). Serial dilutions (10³ to 10⁸) of the retrovirus solution prepared in 2-3 were added to the wells, and after 24 hours the medium was exchanged (D-MEM medium containing 10% FBS to which G418 was added to a final concentration of 1 mg/ml) . Thereafter, selection culture was carried out in the medium containing G418 every other day to check the formation of colonies prior to calculation of titer by counting. Retrovirus vectors having titers of 10⁸ to 10⁹ cfu/ml were obtained from both packaging cell lines pMSCVneoU6NPwpre/GP293 and pMSCVneoU6NPU6M2wpre/GP293.

### 2-5: Production of Transgenic Chicken

Microinjection of retroviruses into fertilized chicken eggs was carried out by the similar manner as described in the patent document (Example 7 of JP 2007-89578 A).

Fertilized chicken eggs (Shiroyama Shukeijo (Shiroyama Chicken Farm); Kakogawa, Japan) were incubated at 38° C. under the condition of 50% or more humidity for 55 hours, during which the eggs were turned by 90 degrees every hour (an incubator from Showa Furanki).

Each of the eggs was opened at the large end with a minirouter equipped with a diamond edge (Proxxon) and the contents were transferred to a double yolk egg (Shiroyama Shukeijo (Shiroyama Chicken Farm); Kakogawa, Japan) which had been cut at the large end for a diameter of 4.5 cm in the same manner as above and the contents of which had been discarded. The embryo was adjusted to be arranged to the upper side, Femtotips II (Eppendorf) were filled with the virus solution under a stereomicroscope system. SZX12 (Olympus) and 2 μl of the retrovirus prepared in 2-3 was microinjected with a microinjector (FemtoJet; Eppendorf).

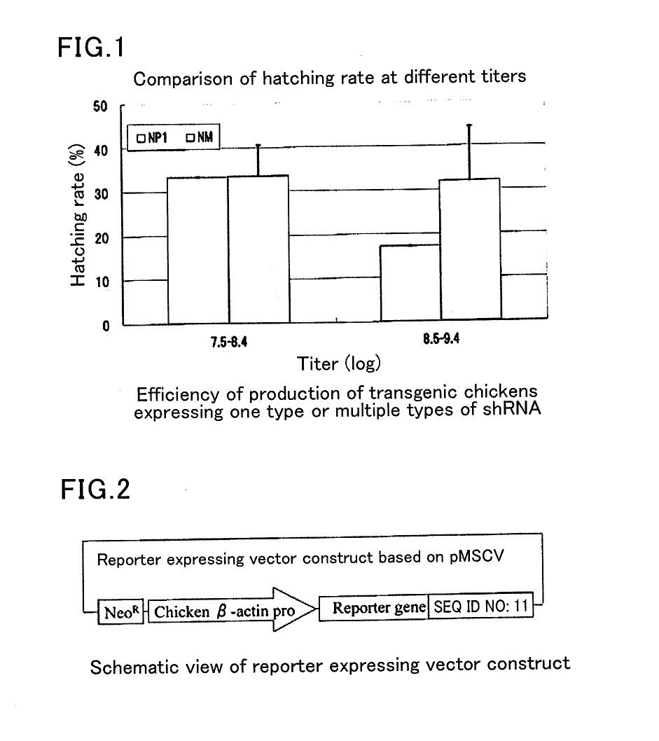

After injection, the openings were covered with Saran Wrap (Asahi Kasei) using egg white as an adhesive and the eggs were re-incubated at 38° C. under the condition of 30-degree egg turning at every hour until hatching (Table 1). Here, titers of viruses introduced (10⁸ cfu/ml or more) and hatching rates are shown in FIG. 1. Generally, it has been said that obtainment of a cell expressing multiple types of functional RNA (such as shRNA) is difficult. However, FIG. 1 surprisingly shows that the hatching rate of the individuals expressing two types of shRNA was similar between with the high titer vector and with the low titer vector. Moreover, when the high titer vector was used, the hatching rate was obviously higher in the individuals expressing two types of shRNA than those expressing one type of shRNA.

TABLE 1

| | Number of | Number of |
|---|---|---|
| Target | injected eggs | hatched eggs |
| One type (NP) | 291 | 60 |
| Multiple types | 410 | 128 |
| (NP and M2) | | |

### 2-6: Detection of Transgene in Transgenic Chicken and Determination of Transgene Copy Number

Whole blood was collected under the wing from each transgenic chicken produced in 2-5. Citric acid was used as an anticoagulant. After flash centrifugation with a mini-centrifuge, a 20 μl supernatant was separated and genomic DNA was extracted with Mag-Extractor Genome kit (TOYOBO). The transgene copy number was calculated with a standard, the genome of the transgenic chicken described in J Virol. 2005 September; 79 (17): 10864-74 (complete transgenic chicken having one copy of a transgene in whole body). Calculation of transgene copy number was carried out by using a real-time PCR instrument (LightCycler; Roche) and a kit (LightCycler FirstStart DNA Master Hybprobe; Roche) with amplification primers (SEQ ID NOs: 5 and 6; SIGMA Genosys) and fluorescence-labeled probes (SEQ ID NOs: 7 and 8; Nihon Gene Research Laboratories) according to the instruction attached to the kit. The SEQ ID NO: 7 is labeled at the 3′-end with FITC and the SEQ ID NO: 8 is labeled at the 5′-end with LCRed640. Real-time PCR was carried out under the following reaction conditions:
Thermal denaturation reaction: 95° C., 10 minutes
Amplification detection reaction: 95° C., 10 seconds→58° C., 15 seconds→72° C., 10 seconds (45 cycles)
Cooling reaction: 40° C.

Transgene copy numbers of the transgenic chickens expressing two types of shRNA (NP and M2) produced in 2-5 are shown in Table 2.

TABLE 2
Average transgene copy number in cells of transgenic chickens

| | Number of chickens |
|---|---|
| Copy number (copies/cell) | expressing two types (NP and M2) |
| 0.00-0.19 | 28 |
| 0.20-0.39 | 34 |
| 0.40-0.59 | 19 |
| 0.60-0.79 | 9 |
| 0.80-0.99 | 7 |
| 1.00-1.99 | 3 |
| 2.00- | 1 |
| Not determined | 5 |
| Total | 128 |

### EXAMPLE 3

### Expression Analysis in Transgenic Chicken

Whole blood was collected under the wing from the transgenic chicken (expressing two types: NP and M2) produced in Example 2 and a wild-type chicken as a negative control. Citric acid was used as an anticoagulant and a total RNA was extracted with Trizol Reagent (Invitrogen). The total RNA of each of these chickens (0.9 ng each) was used as a sample for a reverse transcription reaction, and siRNA expression analysis was carried out with Custom TaqMan Small RNA Assays (Applied Biosystems) and Applied Biosystems 7300 Real-Time PCR System. As a result, two types of siRNA were detected. siRNAs of SEQ ID NOs: 9 and 10 at predetermined concentrations were synthesized (SIGMA Genosys) and used as standards to prepare calibration curves, and the amounts of shRNAs expressed in the transgenic chicken were converted to numbers based on the negative control (Table 3).

TABLE 3
shRNAs expressed in blood of transgenic chicken

| | Copy number (copies/sample) |
|---|---|
| shRNA (NP) | 3,900 |
| shRNA (M2) | 60,000 |

### EXAMPLE 4

### Verification of Anti-Influenza Virus Effect

The inhibitory effect on expression of a target gene caused by expression of shRNAs was verified with chicken embryonic fibroblast (CEF) cells.

### 4-1: Preparation of Reporter Expressing Vector Construct

A synthetic sequence recognized by shRNAs for NP and M2 (SEQ ID NO: 11) was synthesized. The SEQ ID NO: 11 was linked downstream of a reporter gene to construct a reporter expressing vector construct (FIG. 2). The prepared vector construct was used to transform E. coli DH5α (TAKARA), a single colony was cultured in a LB medium (100 ml) while shaking at 37° C. for 16 hours before the cells were collected by centrifugation and purified to be endotoxin-free (EndoFree-plasmid Maxi Kit: QIAGEN).

### 4-2: Preparation of CEF

A sperm chicken egg was incubated for 7 days by the method described in Example 2. The embryo was taken from the incubated sperm egg and the head, internal organs and legs were removed before chopping and addition of trypsin (GIBCO) followed by incubation at 37° C. for 20 minutes. D-MEM High-Glucose (GIBCO) containing 10% FBS was added thereto, centrifugation was carried out, a supernatant was removed, D-MEM was added, and the mixture was left to stand for 3 minutes. A supernatant was then spread on a petri dish and used as CEF.

4-3: Preparation of CFE Expressing shRNA and Reporter Gene

The prepared CEF was seeded on a plate at 1×10⁵ cells and cultured for 24 hrs. The cultured CEF was infected with the pMSCVneoU6NPU6M2wpre retrovirus vector (20 μl) prepared as described in Example 2 and cultured for 24 hrs. The medium was then exchanged to D-MEM to which G418 (SIGMA) was added at a final concentration of 1 mg/ml. Culture was then continued for one week in the medium containing G418. The thus obtained cells were designated as NM/CEF. In the similar manner, three transient retrovirus vectors pMSCVneoU6NPwpre, pMSCVneoU6M2wpre, and pMSCVneoU6GFPwpre were used to prepare three kinds of CEF cells expressing shRNA, NP/CEF, M2/CEF and GFP/CEF.

Each of the thus obtained four kinds of CEFs was seeded in a 6-well plate at 1×10⁵ cells and cultured for 24 hr. The cells were transfected with the reporter expressing vector construct (3 μg) described in 4-1 by using LipofectAMINE 2000. After 6 hours, the medium was exchanged (D-MEM medium containing 10% FBS), 200 μl 1 M HEPES Buffer Solution (GIBCO) was added and culture was continued for additional 24 hours.

### 4-4: Verification of Gene Inhibition Effect

A total RNA was extracted from four kinds of CEFs into which the reporter expressing vector construct was introduced as described in 4-3. Expression of the reporter gene was checked by real-time PCR using specific primers (LightCycler RNA Master SYBR GreenI; Roche). Real-time PCR was carried out under the following conditions:
Reverse transcription reaction: 61° C., 20 minutes
Thermal denaturation reaction: 95° C., 30 seconds
Amplification reaction: 95° C., 5 seconds→55° C., 10 seconds→72° C., 25 seconds (45 cycles)
Cooling: 40° C.

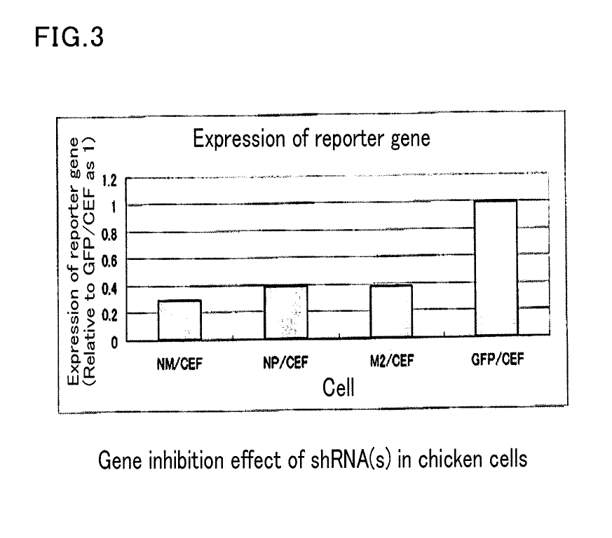

The results are shown in FIG. 3. As can be seen from FIG. 3, expression of the reporter gene was significantly inhibited when two types of functional RNA (shRNA) were expressed, compared to when one type of functional RNA (shRNA) was expressed.

The above examples have been described exemplarily referring to chickens; however, by using the concept of the present invention, other birds and even mammals can be produced which intracellularly express at least two types of functional RNA and have sufficient resistance against a pathogen.

1. A transgenic animal intracellularly expressing at least two types of foreign functional RNA and having resistance to a pathogen.

2. The transgenic animal according to claim 1, wherein the functional RNA is one or more polynucleotides (RNAs) selected from shRNAs, miRNAs, ribozymes, siRNAs and aptamers.

3. The transgenic animal according to claim 1, wherein the functional RNA is a RNA having a base sequence having 50% or more sequence identity with a complementary strand of a partial genomic base sequence of the pathogen.

4. The transgenic animal according to any of claim 1, wherein the resistance to the pathogen corresponds to inhibition of proliferation of the pathogen to 50% or less.

5. The transgenic animal according to claim 1, wherein the pathogen is a virus.

6. The transgenic animal according to claim 5, wherein the virus is an influenza virus.

7. The transgenic animal according to claim 1, wherein the transgenic animal is a bird.

8. The transgenic animal according to claim 7, wherein the bird is a domestic poultry.

9. The transgenic animal according to claim 8, wherein the domestic poultry is a chicken.


SEQUENCE LISTING (11 sequences)

SEQ ID NO: 1 - DNA, 74 bp, Artificial Sequence
  Synthetic polynucleotide
atatggatccccgcaatggactccaacactcttcaagagagagtgttggagtccattgctttttaagcttatat

SEQ ID NO: 2 - DNA, 79 bp, Artificial Sequence
  Synthetic polynucleotide
atatggatccccccacagcagaagctgtggatttcaagagaatccacagcattctgctgttttttggaaaagcttggcc

SEQ ID NO: 3 - DNA, 84 bp, Artificial Sequence
  Synthetic polynucleotide
atatggatccccccggctacgtccaggagcgcatttcaagagaatgcgctcctggacgtagccggtttttggaaaagcttggcc

SEQ ID NO: 4 - DNA, 279 bp, Homo sapiens
aacagaattcaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagtttttaaaattatgttttaaaatggactatcatatgcttaccgtaacttgaaagtatttcgatttcttggctttatatatcttgtggaaaggacgaggatcctccc

SEQ ID NO: 5 - DNA, 21 bp, Artificial Sequence
  Synthetic oligonucleotide
caagaagagacgttgggttac

SEQ ID NO: 6 - DNA, 18 bp, Artificial Sequence
  Synthetic oligonucleotide
cttcccaggtcacgatgt

SEQ ID NO: 7 - DNA, 30 bp, Artificial Sequence
  Synthetic oligonucleotide
ggccaggtgaaaagaccttgatcttaacct

SEQ ID NO: 8 - DNA, 27 bp, Artificial Sequence
  Synthetic oligonucleotide
ggtgatgaggtctcggttaaaggtgcc

SEQ ID NO: 9 - RNA, 20 nt, Artificial Sequence
  Synthetic oligonucleotide
gcaauggacuccaacacucu

SEQ ID NO: 10 - RNA, 18 nt, Artificial Sequence
  Synthetic oligonucleotide
acagcagaaugcugugga

SEQ ID NO: 11 - DNA, 90 bp, Artificial Sequence
  Synthetic polynucleotide
atatacgcgtggaaacaatggactccagcactcttgaacttcaacaggaacagcagagtgctgtggatgttgacgatggtatcgatatat